(19) United States
(12) Patent Application Publication
(10) Pub. No.: US 2026/0077151 A1
(43) Pub. Date: Mar. 19, 2026

(54) DEVICE FOR FACILITATING BRAIN FUNCTION IMPROVEMENT, SYSTEM FOR FACILITATING BRAIN FUNCTION IMPROVEMENT, METHOD FOR FACILITATING BRAIN FUNCTION IMPROVEMENT, PROGRAM FOR FACILITATING BRAIN FUNCTION IMPROVEMENT, AND RECORDING MEDIUM

(71) Applicant: COGSMART Co., Ltd., Tokyo (JP)

(72) Inventors: Yasuyuki TAKI, Tokyo (JP); Akira HIGUCHI, Tokyo (JP); Aizhan IMANKULOVA, Tokyo (JP); Hiroki KATAOKA, Tokyo (JP); Koki KOBAYASHI, Tokyo (JP)

(73) Assignee: COGSMART Co., Ltd., Tokyo (JP)

(21) Appl. No.: 19/108,422

(22) PCT Filed: Sep. 16, 2022
(86) PCT No.: PCT/JP2022/034779

§ 371 Date: Mar. 3, 2025

(51) Int. Cl. A61M 21/00; A63B 24/00; A63B 71/06

(52) U.S. Cl. CPC A61M 21/00; A63B 24/0075; A61M 2205/056; A63B 2024/0068; A63B 2071/0663; A63B 2220/62; A63B 2220/836; A63B 2230/06

(57) ABSTRACT

A brain function improvement facilitating device includes: a storage; a display screen; a timer; a heart rate measurement unit; a target exercise intensity calculator; a setting unit; a first determiner that determines whether a heart rate of a user is greater than or equal to a target heart rate set by the setting unit; a duration time measurement controller that performs control to measure a duration time in which the heart rate of the user is greater than or equal to the target heart rate if the heart rate of the user is determined to be greater than or equal to the target heart rate; a second determiner that determines whether a total of the duration time measured within a predetermined time limit has reached the target time set by the setting unit; a storage controller that controls the storage to store information indicating that the duration time has reached the target time if it is determined that the total of the duration time has reached the target time; a display data generator that generates display data to be displayed on a display screen based on reaching the target time if it is determined that the duration time has reached the target time; and a display controller that controls the display screen to display at least one of the calculated target heart rate, actual related data and time, or the display data.

### TECHNICAL FIELD

The present disclosure relates to a device for facilitating brain function improvement, a system for facilitating brain function improvement, a method for facilitating brain function improvement, a program for facilitating brain function improvement, and a recording medium.

### BACKGROUND ART

Programs for achieving cognitive function improvement are disclosed (for example, Patent Literature 1). Patent Literature 1 describes that a head-mounted display worn by a user displays a viewpoint image of a virtual body of the user in a virtual reality space when the virtual body performs an exercise at a second exercise intensity smaller than a first exercise intensity.

### CITATION LIST

### Patent Literature

Patent Literature 1: Japanese Patent Application Publication No. 2021-81864

### SUMMARY OF INVENTION

### Technical Problem

In recent years, it has been recognized that performing exercise with a certain exercise intensity is beneficial for suppressing or improving declines of brain function, such as slowing the progress of dementia or improving dementia. However, general users often do not know what type of exercise will achieve suppression of declines or improvement of their brain function. Consequently, exercises the user independently perform to suppress decline of or improve brain function might be insufficient or inappropriate. In addition, since users may lack confidence in whether their exercises are suitable or not, they might cease regular exercises. Such situations are undesirable for users seeking to suppress declines of or improve brain function.

It is therefore an object of the present disclosure to provide a device for facilitating brain function improvement capable of easily facilitating decline suppression and improvement of brain function appropriate for users. This brain function improvement facilitating device is also applicable for the purpose of slowing progress of dementia and improving dementia, for example.

### Solution to Problem

A brain function improvement facilitating device according to the present disclosure is a brain function improvement facilitating device attachable to a body of a user and configured to facilitate brain function improvement of the user. The brain function improvement facilitating device includes: a storage that stores data; a display screen that displays data; a timer that measures a time; a heart rate measurement unit that measures a heart rate of the user over time; a target exercise intensity calculator that calculates a target heart rate and a target time, the target heart rate being set as a target exercise intensity necessary for facilitating brain function improvement for the user, the target time being to be reached within a predetermined time limit; a setting unit that sets the target heart rate and the target time calculated by the target exercise intensity calculator; a first determiner that determines whether the heart rate of the user measured by the heart rate measurement unit is greater than or equal to the target heart rate set by the setting unit; a duration time measurement controller that controls the timer to measure a duration time in which the heart rate of the user is greater than or equal to the target heart rate if the first determiner determines that the heart rate of the user is greater than or equal to the target heart rate; a second determiner that determines whether a total of the duration time measured within the predetermined time limit has reached the target time set by the setting unit; a storage controller that controls the storage to store information indicating that the total of the duration time has reached the target time if the second determiner determines that the total of the duration time has reached the target time; a display data generator that generates display data to be displayed on the display screen based on reaching the target time if the second determiner determines that the total of the duration time has reached the target time; and a display controller that controls the display screen to display at least one of the target heart rate calculated by the target exercise intensity calculator, actual related data and time, or the display data generated by the display data generator.

### Advantageous Effects of Invention

The brain function improvement facilitating device described above can easily facilitate brain function improvement appropriate for the user.

### BRIEF DESCRIPTION OF DRAWINGS

FIG. 1 schematically illustrates an appearance of a brain function improvement facilitating device according to a first embodiment.

FIG. 2 is a block diagram illustrating a configuration of the brain function improvement facilitating device illustrated in FIG. 1.

FIG. 3 is a flowchart depicting typical process steps in calculating and setting a target exercise intensity.

FIG. 4 is a flowchart depicting typical process steps in facilitating brain function improvement with the brain function improvement facilitating device according to the first embodiment.

FIG. 5 is a schematic view illustrating an example of display data displayed on a display screen.

FIG. 6 is a block diagram illustrating a brain function improvement facilitating system according to a second embodiment.

FIG. 7 is a schematic view illustrating an example of display data displayed on a display screen.

FIG. 8 is a schematic view illustrating an example of display data displayed on the display screen.

### DESCRIPTION OF EMBODIMENTS

### Description of Embodiments of the Present Disclosure

First, embodiments of the present disclosure will be listed and described. A device for facilitating brain function improvement according to the present disclosure is a brain function improvement facilitating device attachable to a body of a user and configured to facilitate improvement of brain function of the user. A brain function improvement facilitating device includes: a storage that stores data; a display screen that displays data; a timer that measures a time; a heart rate measurement unit that measures a heart rate of the user over time; a target exercise intensity calculator that calculates a target heart rate and a target time, the target heart rate being set as a target exercise intensity necessary for facilitating brain function improvement for the user, the target time being to be reached within a predetermined time limit; a setting unit that sets the target heart rate and the target time calculated by the target exercise intensity calculator; a first determiner that determines whether the heart rate of the user measured by the heart rate measurement unit is greater than or equal to the target heart rate set by the setting unit; a duration time measurement controller that controls the timer to measure a duration time in which the heart rate of the user is greater than or equal to the target heart rate if the first determiner determines that the heart rate of the user is greater than or equal to the target heart rate; a second determiner that determines whether a total of the duration time measured within the predetermined time limit has reached the target time set by the setting unit; a storage controller that controls the storage to store information indicating that the total of the duration time has reached the target time if the second determiner determines that the total of the duration time has reached the target time; a display data generator that generates display data to be displayed on the display screen based on reaching the target time if the second determiner determines that the total of the duration time has reached the target time; and a display controller that controls the display screen to display at least one of the target heart rate calculated by the target exercise intensity calculator, actual related data and time, or the display data generated by the display data generator.

Inventors of the present disclosure have focused on effectiveness of exercises with a certain level of intensity in, for example, suppressing declines of brain function of a user or improvement of brain function of the user including suppression of progress of dementia, improvement of cognitive abilities, or the like. In view of this, users may attempt to engage in prolonged exercises such as running to improve brain function. Running herein is an example of an exercise with a certain exercise intensity. Aerobic exercises such as monotonous running are relatively strenuous, and it is often difficult to sustain such exercises. Moreover, the inventors of the present disclosure have considered that, in many cases, even without engaging in running with such a high exercise intensity, consistency can be effective for suppressing declines of brain function, improve brain function, and others. Therefore, the inventors considered that it is important to clearly define a target exercise intensity necessary or desirable for suppressing a decline of brain function or improving brain function when engaging an exercise. For ease of explanation, suppression of a decline of brain function will be herein included in the concept of improvement of brain function. Here, the inventors of the present disclosure focused on the fact that exercises satisfying the target exercise intensity can be found in daily activities, even without conscious engagement of users in strenuous exercises such as running. For example, the inventors further focused on the fact that activities such as walking a dog, brisk walking during a commute, or climbing stairs may satisfy the target exercise intensity. The inventors hypothesized that raising awareness of users that these everyday activities (regardless of whether the activities are aerobic or anaerobic) can improve brain function would lead to sustained engagement in exercises satisfying the target exercise intensity, and consequently would effectively facilitate improvement of brain function in the case of intentional exercise for improvement of brain function and the case of exercises for other health maintenance or improvement or in daily routines without such special awareness.

The inventors of the present disclosure further considered as follows. An exercise that satisfies the target exercise intensity necessary or desirable for the user to facilitate brain function improvement is a balance between a target heart rate and a target time. That is, although the target heart rate can be set with a certain range while being set according to the attribute of the user or the like, the higher the target heart rate is, the higher the effects for facilitating brain function improvement are. On the other hand, since an exercise with a higher target heart rate is more strenuous, it may be difficult for some users to perform the exercise continuously or without interruption. If it becomes difficult to perform the exercise continuously or without interruption, or if the exercise is not continued in some cases, the effects of brain function improvement decrease, or the exercise may be meaningless.

With the brain function improvement facilitating device described above, the brain function improvement facilitating device attached to the body of a user measures a heart rate of the user over time, measures a duration time in which the heart rate is greater than or equal to the target heart rate, and when the measured time reaches a target time to be reached within a predetermined time limit, displays information indicating that the measured time has reached the target time on a display screen. In addition, by activating the brain function improvement facilitating device, it is also possible to calculate a target heart rate and a target time set as a target exercise intensity necessary or desirable for the user to facilitate brain function improvement, and display the target exercise intensity on the display screen. This makes it possible to visually recognize on the display screen that an exercise with a certain exercise intensity that leads to improvement of brain function in a daily life has been performed. Accordingly, the user can be aware that an exercise with a certain exercise intensity that leads to improvement of brain function is performed in a daily life without difficulty. That is, even if the user does not actively go to a sport gym or other places to perform a certain exercise such as running, the user can be aware that the user is performing an exercise with a certain exercise intensity that leads to brain function improvement. The brain function improvement facilitating device is, of course, utilized even in cases where the user intentionally exercises to improve brain function and where the user exercises to maintain or improve other health conditions. Specifically, in performing such an exercise, to clarify a target exercise intensity necessary or desirable for facilitating brain function improvement, the device may calculate a target heart rate and a target time to display the target exercise intensity on the display screen. Accordingly, the user can continuously perform an exercise that leads to brain function improvement without performing an excessively strenuous exercise. Further, since the user can grasp that the user has performed a certain exercise effective for improving brain function by the display on the display screen, even in a daily life without such special consciousness, the user can facilitate brain function improvement by trying to perform an exercise satisfying the target exercise intensity. In this manner, the brain function improvement facilitating device described above can easily facilitate brain function improvement appropriate for the user.

In the brain function improvement facilitating device, if the second determiner does not determine that the duration time has not reached the target time, the second determiner may determine whether the duration time has reached a first set time shorter than the target time. If the second determiner determines that the duration time has reached the first set time, the storage controller may control the storage to store information indicating that the duration time has reached the first set time. If the second determiner determines that the duration time has reached the first set time, the display data generator may generate display data to be displayed on the display screen based on reaching the first set time. With this configuration, even if the duration time does not reach the target time, as long as the duration time has reached the first set time, the user can easily visually recognize that the duration time has reached the first set time. Thus, the user can facilitate brain function improvement by trying to perform the same constant exercise in a daily life continuously or without interruption to approach the target time.

In the brain function improvement facilitating device, if the second determiner determines that the duration time has reached the first set time, the storage controller may control the storage to store data of the duration time. With this configuration, even if the duration time does not reach the target time, the duration time is added and displayed, for example, so that it is possible to visually appeal that the user has performed an effective exercise leading to brain function improvement. This can lead to facilitating brain function improvement through an exercise within a predetermined time limit.

In the brain function improvement facilitating device, the brain function improvement may include at least one of suppression of progress of dementia, suppression of atrophy of brain including a negative change of brain morphology, or improvement of a brain health score. The certain exercise described above is significantly effective in suppressing the progress of dementia, suppressing atrophy of brain morphology including a negative change in brain morphology, and improving brain health scores.

In the brain function improvement facilitating device, if the first determiner does not determine that the heart rate of the user measured by the heart rate measurement unit is greater than or equal to the target heart rate within a certain period of the time measured by the timer from a predetermined timing, the display data generator may generate display data indicating that the first determiner does not determine that the heart rate of the user measured by the heart rate measurement unit is greater than or equal to the target heart rate. This configuration makes it possible to visually appeal to the user that the user does not perform an exercise for a certain period of time so that the heart rate is greater than or equal to the target heart rate. Accordingly, it is possible to facilitate the user to perform an exercise at a heart rate greater than or equal to the target heart rate in a daily life.

In the brain function improvement facilitating device, determination by the first determiner may be refreshed at each predetermined timing. This configuration makes it possible to effectively facilitate repetitive certain exercises for a short period such as one week or a long period such as several months.

In the brain function improvement facilitating device, the target exercise intensity calculator may calculate the target heart rate using at least one of a rule-based model, a machine learning model, or a deep learning model. This makes it possible to calculate a target heart rate that is more meaningful for the user, and to easily facilitate brain function improvement that is more appropriate for the user. In this case, the target heart rate may be calculated using any one of the rule-based model, the machine learning model, and the deep learning model, may be calculated using a method combining the rule-based model and the machine learning model, a method combining the rule-based model and the deep learning model, or a method combining the machine learning model and the deep learning model, or may be calculated using a method using the three models of the rule-based model, the machine learning model, and the deep learning model. The target heart rate may be, of course, calculated using a method combining any one of the rule-based model, the machine learning model, and the deep learning model with another method.

In the brain function improvement facilitating device, the target exercise intensity calculator may calculate, as the target heart rate, an optimized target heart rate that is a heart rate as high as possible while satisfying a condition of performing an exercise continuously or without interruption.

To maximize the effects of brain function improvement for the user, it is useful to set the target heart rate as high as possible while satisfying the condition of performing an exercise continuously or without interruption. Such setting of the target heart rate varies among individual users, depending on factors such as the user's age, gender, exercise experience (any of short-term, medium-term, or long-term exercises), physical ability, and motivation.

In view of this, the inventors of the present disclosure considered that “setting the target heart rate as high as possible while satisfying a condition of performing an exercise continuously or without interruption” is effective for maximizing the effects of improving brain function, and a method for determining a target heart rate suitable for each user depending on factors such as the age, gender, exercise experience, physical ability, and motivation of the user. This is applicable to the brain function improvement facilitating device, and to any of a brain function improvement facilitating system, a brain function improvement facilitating method, a brain function improvement facilitating program, and a recording medium described later.

The “target heart rate as high as possible while satisfying the condition of performing an exercise continuously or without interruption” is defined as an “optimized target heart rate.”

An optimized target heart rate of a user is calculated from a target exercise intensity calculator by inputting qualitative or quantitative information including (1) and (2):
(1) Information obtained by the target exercise intensity calculator is (a) a target heart rate of a user.
(2) Information obtained by input from the user is (a) a target time of an exercise in the case of exercising from now on and (b) a target or preference level of the user (hereinafter referred to as a “user preference” and may be represented as “Pi”) with respect to an exercise intensity of the exercise.

The user preference (Pi) may be represented as an objective numerical value (e.g., a percentage notation such as “80%” in the case of exercising at full strength) serving as a plurality of options from the target exercise intensity calculator. Alternatively, the user preference (Pi) may be selected from a plurality of subjective options (e.g., five levels of highest, high, middle, low, easy, or the like) by the user. In this case, the target exercise intensity calculator converts the subjective options into appropriate objective numerical values by a predetermined calculation formula.

As information for calculating the optimized target heart rate, information other than the information (1) and (2) may be input. For example, an age or gender of the user may be input from the target exercise intensity calculator, a sleep time last night and other healthcare data may be input from the device, or evaluation information regarding physical conditions of the day may be input from the user.

The optimized target heart rate is a heart rate satisfying all of (3), (4), and (5):
(3) The heart rate is greater than or equal to a heart rate necessary for facilitating brain function improvement for the user.
(4) The heart rate is a heart rate at which an exercise can be performed with a constant heart rate in a duration time (expressed as X minutes, for example, 35 minutes). The “constant” herein is a concept having a certain degree of width, and refers to, for example, keeping a heart rate corresponding to a range from 95% or more and 105% or less of a heart rate set as an optimized target heart rate. For example, in a case where the optimized target heart rate is 80, the heart rate is 76 or more and 84 or less is referred to as constant.
(5) The heart rate is obtained after multiplying the “exercise intensity” in the calculation formula of the “target heart rate HRt” by an “objective numerical value” conforming to the user preference (Pi).

As the calculation formula model constituting the target exercise intensity calculator and used for obtaining an optimized target heart rate, various methods such as the machine learning model and the deep learning model as well as the rule-based model can be used.

In the case of the rule-based model, a calculation rule of the optimized target heart rate is defined based on research results and knowledge of existing sports medicine, science, and the like, and the optimized target heart rate is predicted by the calculation rule.

In the case of the machine learning model, various methods such as a support vector machine (SVM), decision trees, a random forest, and a K-nearest neighbor can be used. In the case of the deep learning model, various methods such as a multi-layer neural network (MNN), a recurrent neural network (RNN), a long short-term memory network (LSTM), a convolutional neural network (CNN), and a transformer can be used. These methods optimize parameters of a model by performing supervised learning with given data including an input and an output, and output an “optimized target heart rate” that is a target heart rate as high as possible while satisfying a condition of performing an exercise continuously or without interruption.

The formula constituting the target exercise intensity calculator and used for obtaining the optimized target heart rate is leaned for optimizing the target heart rate of the user using feedback from the user as a reward by reinforcement leaning, and is changed accordingly. That is, in a case where the user actually exercises after obtaining the optimized target heart rate, the following results can be obtained.
(6) Duration time of the exercise
(7) Volatility of a heart rate with a target heart rate as an axis during the exercise
(8) User's subjective evaluation on a presented exercise intensity (target heart rate) (if possible)

The calculation formula for obtaining an optimized target heart rate is not limited to a single method among the rule-based model, the machine learning model, and the deep learning model described above, and a further optimized output of the user can be performed by combining a plurality of models.

Through the foregoing process, the formula constituting the target exercise intensity calculator and used for obtaining the optimized target heart rate is learned for each user constantly or as appropriate. In this manner, it is possible to achieve the original purpose of “maximizing the effects of brain function improvement” by “setting the target heart rate as high as possible while satisfying the condition of performing an exercise continuously or without interruption” for each user.

A leaning procedure of the leaning model is performed in any case based on the following approach.
(A) Prepare the i-th input xi.
(B) Give the input to a model F (·; θ) having a parameter θ, and calculate a prediction yi=F (xi; θ) for the i-th data.
(C) Calculate an error between the prediction yi and a correct answer yi of the i-th data.
(D) Update the parameter θ of the model based on the error.
(E) Perform the steps (A) through (D) for all the leaning data until the leaning of the model converges in development data.

A system for facilitating brain function improvement according to the present disclosure is a brain function improvement facilitating system that facilitates brain function improvement and includes: a measurement sensor attachable to a body of a user and including a heart rate measurement unit that measures a heart rate of the user over time; and a terminal device communicable with the measurement sensor. The terminal device includes a storage that stores data, a display screen that displays data, a timer that measures a time, a target exercise intensity calculator that calculates a target heart rate and a target time, the target heart rate being set as a target exercise intensity necessary for facilitating brain function improvement for the user, the target time being to be reached within a predetermined time limit, a setting unit that sets the target heart rate and the target time calculated by the target exercise intensity calculator, a first determiner that determines whether the heart rate of the user measured by the heart rate measurement unit is greater than or equal to the target heart rate set by the setting unit, a duration time measurement controller that controls the timer to measure a duration time in which the heart rate of the user is greater than or equal to the target heart rate if the first determiner determines that the heart rate of the user is greater than or equal to the target heart rate, a second determiner that determines whether a total of the duration time measured within the predetermined time limit has reached the target time set by the setting unit, a storage controller that controls the storage to store information indicating that the duration time has reached the target time if the second determiner determines that the duration time has reached the target time, a display data generator that generates display data to be displayed on the display screen based on reaching the target time if the second determiner determines that the total of the duration time has reached the target time, and a display controller that controls the display screen to display at least one of the target heart rate calculated by the target exercise intensity calculator, actual related data and time, or the display data generated by the display data generator.

This brain function improvement facilitating system can easily facilitate improvement of brain function appropriate for the user.

A brain function improvement facilitating method according to the present disclosure is a brain function improvement facilitating method for facilitating improvement of brain function of a user and includes: measuring a heart rate of the user over time; calculating a target heart rate and a target time, the target heart rate being set as a target exercise intensity necessary for facilitating brain function improvement for the user, the target time being to be reached within a predetermined time limit; setting the calculated target heart rate and target time; determining whether the measured heart rate of the user is greater than or equal to the target heart rate; performing control to measure a duration time in which the heart rate of the user is greater than or equal to the target heart rate if it is determined that the heart rate of the user is greater than or equal to the target heart rate; determining whether a total of the duration time measured within the predetermined time limit has reached the target time; performing control to store information indicating that the duration time has reached the target time if it is determined that the duration time has reached the target time; generating display data to be displayed based on reaching the target time if it is determined that the total of the duration time has reached the target time; and performing control to display at least one of the calculated target heart rate, actual related data and time, or the generated display data.

This brain function improvement facilitating method can easily facilitate improvement of brain function appropriate for the user.

A brain function improvement facilitating program according to the present disclosure is a program for a brain function improvement facilitating system for use in a brain function improvement facilitating system including a measurement sensor attachable to a body of a user and including a heart rate measurement unit that measures a heart rate of the user over time, and a terminal device including a storage that stores data, a display screen that displays data, and a timer that measures a time, the terminal device is communicable with the measurement sensor, and the program causes the terminal device to function as: a target exercise intensity calculator that calculates a target heart rate and a target time, the target heart rate being set as a target exercise intensity necessary for facilitating brain function improvement for the user, the target time being to be reached within a predetermined time limit; a setting unit that sets the target heart rate and the target time calculated by the target exercise intensity calculator; a first determiner that determines whether the heart rate of the user measured by the heart rate measurement unit is greater than or equal to the target heart rate set by the setting unit; a duration time measurement controller that controls the timer to measure a duration time in which the heart rate of the user is greater than or equal to the target heart rate if the first determiner determines that the heart rate of the user is greater than or equal to the target heart rate; a second determiner that determines whether a total of the duration time measured within the predetermined time limit has reached the target time set by the setting unit; a storage controller that controls the storage to store information indicating that the duration time has reached the target time if the second determiner determines that the duration time has reached the target time; a display data generator that generates display data to be displayed on the display screen based on reaching the target time if the second determiner determines that the total of the duration time has reached the target time; and a display controller that controls the display screen to display at least one of the target heart rate calculated by the target exercise intensity calculator, actual related data and time, or the display data generated by the display data generator.

This brain function improvement facilitating program can easily facilitate improvement of brain function appropriate for the user.

A recording medium according to the present disclosure is a computer-readable recording medium for use in a brain function improvement facilitating system that facilitates improvement of brain function of a user and includes a measurement sensor attachable to a body of a user and including a heart rate measurement unit that measures a heart rate of the user over time, and a terminal device including a storage that stores data, a display screen that displays data, and a time that measures a time, the terminal device is communicable with the measurement sensor, and the recording medium causes the terminal device to function as: a target exercise intensity calculator that calculates a target heart rate and a target time, the target heart rate being set as a target exercise intensity necessary for facilitating brain function improvement for the user, the target time being to be reached within a predetermined time limit; a setting unit that sets the target heart rate and the target time calculated by the target exercise intensity calculator; a first determiner that determines whether the heart rate of the user measured by the heart rate measurement unit is greater than or equal to the target heart rate set by the setting unit; a duration time measurement controller that controls the timer to measure a duration time in which the heart rate of the user is greater than or equal to the target heart rate if the first determiner determines that the heart rate of the user is greater than or equal to the target heart rate; a second determiner that determines whether a total of the duration time measured within the predetermined time limit has reached the target time set by the setting unit; a storage controller that controls the storage to store information indicating that the duration time has reached the target time if the second determiner determines that the duration time has reached the target time; a display data generator that generates display data to be displayed on the display screen based on reaching the target time if the second determiner determines that the total of the duration time has reached the target time; and a display controller that controls the display screen to display at least one of the target heart rate calculated by the target exercise intensity calculator, actual related data and time, or the display data generated by the display data generator.

This storage medium can easily facilitate improvement of brain function appropriate for the user.

### Details of Embodiments of the Present Invention

Embodiments of the brain function improvement facilitating device according to the present disclosure will be described with reference to the drawings. In the following drawings, the same or corresponding parts are denoted by the same reference numerals, and the description thereof will not be repeated.

### First Embodiment

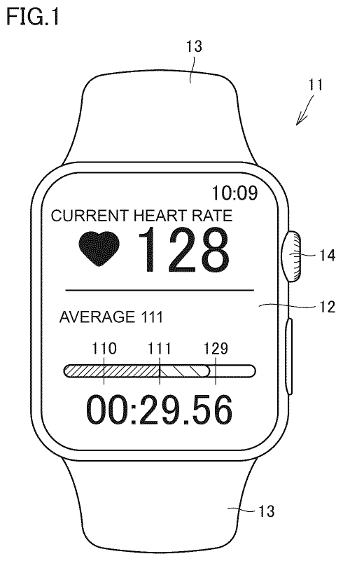

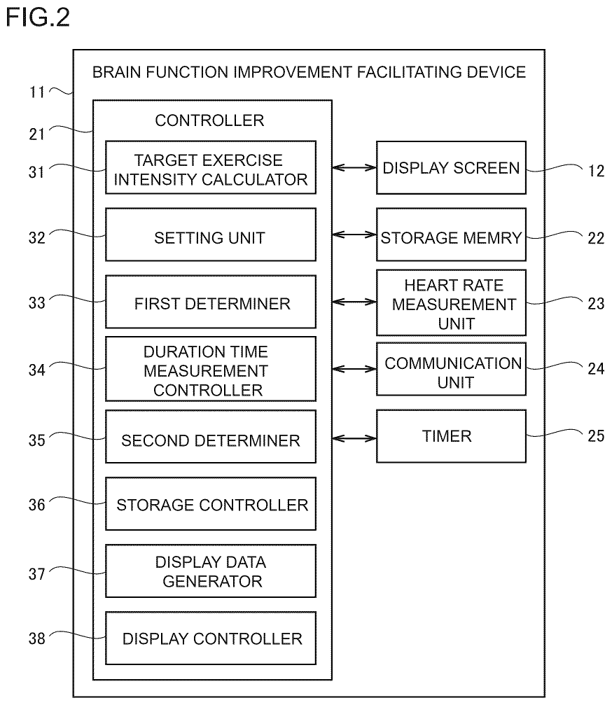

A configuration of a brain function improvement facilitating device according to a first embodiment of the present disclosure will be described. FIG. 1 schematically illustrates an appearance of the brain function improvement facilitating device according to the first embodiment. FIG. 2 is a block diagram illustrating a configuration of the brain function improvement facilitating device illustrated in FIG. 1

With reference to FIGS. 1 and 2, a brain function improvement facilitating device 11 according to the first embodiment is detachably attached to the body of a user. That is, the user can attach and detach the brain function improvement facilitating device 11 according to the first embodiment to and from the body of the user. The brain function improvement facilitating device 11 is, for example, a wristwatch type, and can be worn by being wound around an arm of the user. The device may be of a type other than a wristwatch type, and may be of, for example, a wrist band type or a ring type.

The brain function improvement facilitating device 11 includes a display screen 12 that displays data, and a band 13 that is used when the device is wound around an arm of the user. When the brain function improvement facilitating device 11 is wound around the arm of the user with the band 13, the display screen 12 is positioned at a position where the display screen 12 can be visually recognized by the user. The brain function improvement facilitating device 11 further includes buttons 14 for various settings located on a side surface of the display screen 12.

The brain function improvement facilitating device 11 includes a controller 21 that controls the brain function improvement facilitating device 11 itself, a storage memory 22 as a storage, a heart rate measurement unit 23 that measures a heart rate of the user over time, a communication unit 24 that performs communication between the brain function improvement facilitating device 11 and the outside, and a timer 25 that measures a time. Since the brain function improvement facilitating device 11 includes the timer 25, the brain function improvement facilitating device 11 can also function as a simple clock. The heart rate measurement unit 23 measures a heartbeat or a pulse from the arm of the user, and measures a numerical value of the heartbeat or the pulse as a heart rate.

The controller 21 includes a target exercise intensity calculator 31, a setting unit 32, a first determiner 33, a duration time measurement controller 34, a second determiner 35, a storage controller 36, a display data generator 37, and a display controller 38. The target exercise intensity calculator 31 calculates a target heart rate set as a target exercise intensity necessary for facilitating brain function improvement for the user, and a target time to be reached within a predetermined time limit. The setting unit 32 sets the target heart rate and the target time calculated by the target exercise intensity calculator 31. The first determiner 33 determines whether the heart rate of the user measured by the heart rate measurement unit 23 is greater than or equal to the target heart rate set by the setting unit 32. If the first determiner 33 determines that the heart rate of the user is greater than or equal to the target heart rate, the duration time measurement controller 34 controls the timer 25 to measure a duration time in which the heart rate of the user is greater than or equal to the target heart rate. The second determiner 35 determines whether a total of the duration time measured within a predetermined time limit has reached the target time set by the setting unit 32. If the second determiner 35 determines that the duration time has reached the target time, the storage controller 36 controls the storage to store information indicating that the duration time has reached the target time. If the second determiner 35 determines that the total of the duration time has reached the target time, the display data generator 37 generates display data to be displayed on the display screen 12 based on reaching the target time. The display controller 38 controls the display screen 12 to display at least one of the target heart rate generated by the target exercise intensity calculator 31, actual related data and time, or the display data generated by the display data generator 37. These configurations will be described in detail later.

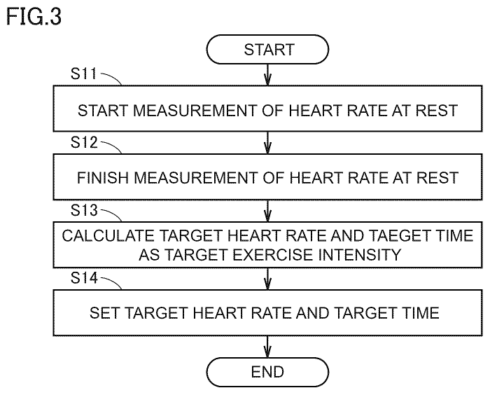

Next, a case where the target exercise intensity is calculated, and set will be described. FIG. 3 is a flowchart depicting typical process steps in calculating and setting a target exercise intensity. Referring to FIG. 3, first, the user wears the brain function improvement facilitating device 11 on an arm of the user. Then, the user maintains a resting state, that is, a state in which the user does not exercise. Here, the brain function improvement facilitating device 11 starts measurement of a heart rate at rest by pressing a switch or the like (step S11 in FIG. 3, “step” will be hereinafter omitted). After a lapse of a predetermined time, the measurement of the heart rate at rest is finished (S12). Then, the target exercise intensity calculator 31 calculates a target heart rate and a target time (S13).

Examples of the target heart rate include the following: First, the heart rate measurement unit 23 measures a heart rate at rest. The heart rate at rest refers to a heart rate in a state where the user is not exercising. The heart rate at rest may be obtained by an input from the user. The heart rate at rest varies among users. A value calculated by the following equation is a target heart rate HRt.

$$target heart rate HRt = exercise intensity \times ( maximum heart rate - heart rate at rest ) + heart rate at rest$$

The maximum heart rate is a maximum value of a heart rate that can be exhibited in one minute. The maximum heart rate may be derived by using any of the following expressions, for example, or a largest heart rate obtained by inputting a numerical value from the user or actually measured within a predetermined time limit may be acquired as the maximum heart rate.

$$maximum heart rate = 220 - age$$

$$maximum heart rate = 210 - 0.5 \times age$$

$$maximum heart rate = 208 - 0.7 \times age$$

Here, the exercise intensity is a numerical value set by the user, and is represented by a percentage, and for example, a value such as 50% or 60% is selected. That is, the target heart rate takes a value between the maximum heart rate and the heart rate at rest.

The target time to be reached within a predetermined time limit is set to be 120 minutes or 150 minutes per week, for example. The target time does not need to be achieved by a single exercise, and rather it is often desirable to achieve a balanced and continuous exercise through a week. In this case, the duration time that has reached the predetermined set time is accumulated multiple times, and thus, reaches the target time such as 120 minutes or 150 minutes. For the purpose of brain function improvement, one type of the set time of 60 minutes may be prepared as a single set time. For example, if an exercise exceeding 60 minutes is performed twice a week, the exercise is accumulated to 120 minutes, and 120 minutes that is the target time in a week is achieved with the condition satisfied. As for the set time, for the purpose of brain function improvement, two types of set times, that is, 30 minutes as a first set time and 5 minutes as a second set time may be prepared. For example, when the user performs a 30-minute exercise (that reaches the second set time and the first set time) twice in a week and performs a 5-minute exercise (that does not reach the first set time but reaches the second set time) 12 times, the exercise time is 120 minutes in total, which achieves 120 minutes that is the target time in one week with the condition satisfied. The target heart rate and the target time set as the target exercise intensity may be displayed on the display screen described later.

The target exercise intensity calculator 31 may calculate, as a target heart rate, an optimized target heart rate that is a target heart rate as high as possible while satisfying a condition of performing an exercise continuously or without interruption. That is, the target exercise intensity calculator 31 may calculate, as a target heart rate, an optimized target heart rate that is a heart rate as high as possible while satisfying a condition of performing an exercise continuously or without interruption. The optimized target heart rate may be calculated using at least one of the rule-based model, the machine learning model, or the deep learning model described above.

Thereafter, the target heart rate and the target time are set by the setting unit 32.

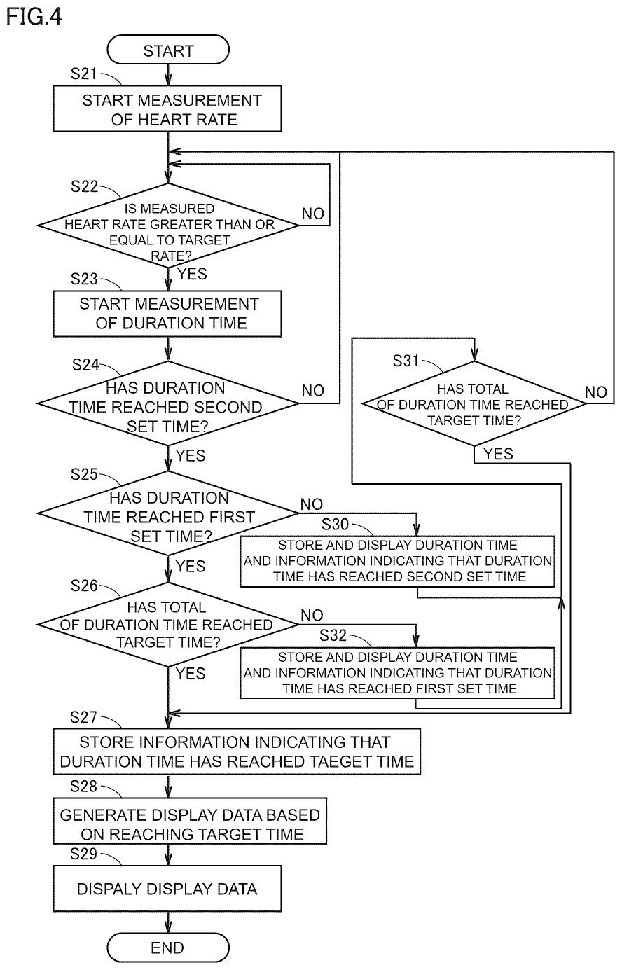

Next, in a situation where the target exercise intensity is set, operation of the brain function improvement facilitating device 11 as described above will be described. FIG. 4 is a flowchart depicting typical process steps in facilitating brain function improvement with the brain function improvement facilitating device 11 according to the first embodiment. As the set time, for the purpose of brain function improvement, one type of the set time may be prepared as a single set time or two or more types of set times may be prepared. In FIG. 4, two types of set times, the first set time and the second set time, are set.

Referring to FIG. 4, when the user wears the brain function improvement facilitating device 11 on an arm of the user, the heart rate measurement unit 23 included in the brain function improvement facilitating device 11 detects a heart rate of the user. Then, the heart rate measurement unit 23 measures the heart rate of the user over time (S21). That is, the heart rate measurement unit 23 starts measuring the heart rate of the user over time. In this case, the measurement of the heart rate of the user by the heart rate measurement unit 23 may be started by detecting pressing of the button 14 included in the brain function improvement facilitating device 11. The brain function improvement facilitating device 11 may include a mechanism that detects a body temperature such that when the mechanism detects the body temperature when the device is worn by the user, the mechanism causes the heart rate measurement unit 23 to start measurement of the heart rate.

After the start of measurement of the heart rate, the user goes about daily activities while wearing the brain function improvement facilitating device 11 on the arm. In this case, the user performs an exercise to move the body of the user, such as walking a dog, doing household work, or doing garden work. This daily exercise does not include running and swimming recognized as an aerobic exercise by the user. For example, assume that the user performs an exercise of continuing to work for a long time with a dog while walking the dog. The first determiner 33 determines whether the heart rate of the user measured by the heart rate measurement unit 23 is greater than or equal to the target heart rate set for each user (S22).

If the first determiner 33 determines that the heart rate of the user is greater than or equal to the target heart rate (YES in S22), the duration time measurement controller 34 controls the timer 25 to measure a duration time in which the heart rate of the user is greater than or equal to the target heart rate (S23).

Then, the user continues to walk the dog. Accordingly, the heart rate greater than or equal to the target heart rate continues. Here, the second determiner 35 determines whether the measured duration time has reached the predetermined second set time or not (S24). The second set time is a certain continuous time that is considered to be minimally effective for improving brain function, and a numerical value such as 5 minutes (300 seconds) is selected, for example. The second set time is shorter than the first set time described later.

If the second determiner 35 determines that the duration time has reached the second set time, the heart rate is continuously measured thereafter. Thue user continues to walk the dog.

Subsequently, the second determiner 35 determines whether the duration time has reached the first set time longer than the second set time (S25). The first set time is a time recognized as a certain exercise considered to be effective for improving brain function in a series of continuous exercises, and a numerical value such as 30 minutes (1800 seconds) is selected, for example.

If the second determiner 35 determines that the duration time has reached the first set time (YES in S25), the second determiner 35 determines whether a total of the duration time measured within a predetermined time limit has reached the target time (S26). In this case, if the second determiner 35 determines that the total of the duration time measured within the predetermined time limit reaches the target time (YES in S26), control is performed such that information indicating the total of the duration time has reached the target value is stored in the storage memory 22 (S27).

Thereafter, the display data generator 37 generates display data to be displayed on the display screen 12 and indicating that the second determiner 35 determines that the total of the duration time has reached the target time (S28). Then, the display controller 38 controls the display screen 12 to display the display data generated by the display data generator 37 (S29).

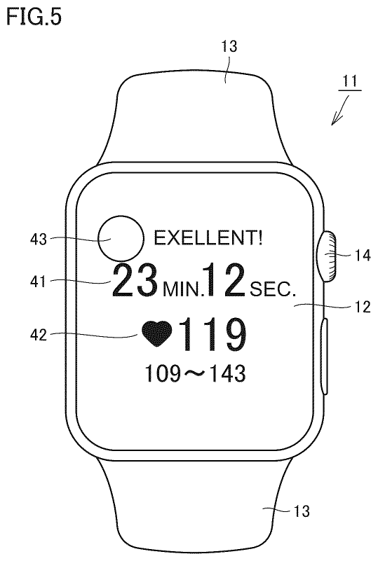

FIG. 5 is a schematic view illustrating an example of display data displayed on the display screen 12. Referring to FIG. 5, the display screen 12 displays display data 41 of a duration time that is a time in which a heart rate greater than or equal to the target heart rate is measured, display data 42 of a heart rate that is an average of measured heart rates, and display data 43 of characters “Excellent” that is a word of a reward for reaching the target time. When the user returns from dog walking, the user can visually recognize the data 41, 42, and 43 visualized on the display screen 12.

With the brain function improvement facilitating device 11 according to the present disclosure, the brain function improvement facilitating device 11 attached to the body of the user measures a heart rate of the user over time, measures the duration time in which the heart rate of the user is greater than or equal to the target heart rate, and when the measured time reaches the target time to be reached within the predetermined time limit, displays that the measured time has reached the target time on the display screen 12. In addition, by activating the brain function improvement facilitating device 11, it is also possible to calculate a target heart rate and a target time set as a target exercise intensity necessary or desirable for the user to facilitate brain function improvement, and display the target exercise intensity on the display screen 12. This makes it possible to visually recognize on the display screen 12 that an exercise with a certain exercise intensity that leads to improvement of brain function in a daily life has been performed. Accordingly, the user can be aware that an exercise with a certain exercise intensity that leads to improvement of brain function is performed in a daily life without difficulty. That is, even if the user does not actively go to a sport gym or other places to perform a certain exercise such as running, the user can be aware that the user is performing an exercise with a certain exercise intensity that leads to brain function improvement. The brain function improvement facilitating device 11 is, of course, utilized even in cases where the user intentionally exercises to improve brain function and where the user exercises to maintain or improve other health conditions. Specifically, in performing such an exercise, to clarify a target exercise intensity necessary or desirable for facilitating brain function improvement, the device may calculate a target heart rate and a target time to display the target exercise intensity on the display screen 12. In this manner, the user can perform an exercise that leads to brain function improvement continuously without performing an excessively strenuous exercise. Further, since the user can grasp that the user has performed a certain exercise effective for improving brain function by the display on the display screen 12, even in a daily life without such special consciousness, the user can facilitate brain function improvement by trying to perform an exercise satisfying the target exercise intensity. In this manner, the brain function improvement facilitating device 11 described above can easily facilitate brain function improvement appropriate for the user.

In S24, if the second determiner 35 does not determine that the duration time has reached the second set time (NO in S24), the process returns to S22, and it is determined whether the measured heart rate is greater than or equal to the target heart rate. In S25, if the second determiner 35 does not determine that the duration time has reached the first set time (NO in S25), the storage controller 36 controls the storage memory 22 to store information indicating that the duration time has reached the second set time and data of the duration time (S30). In this case, the total of the duration time may be displayed. Then, the second determiner 35 determines whether a total of the duration time in a predetermined time limit has reached the target time (S31). If it is not determined that the duration time has reached the target time (NO in S31), the process returns to S22, and it is determined whether the measured heart rate is greater than or equal to the target heart rate. On the other hand, if it is determined that the duration time has reached the target time (YES in S31), controls is performed such that information indicating that the duration time has reached the target time is stored in the storage memory 22 (S27). Thereafter, the display data controller 37 generates display data to be displayed on the display screen 12 and indicating that the second determiner 35 determines that the total of the duration time has reached the target time (S28). Then, the display controller 38 performs the display screen 12 to display the display data generated by the display data generator 37 (S29).

In S26, if the second determiner 35 does not determine that the total of the duration time has reached the target time (NO in S26), the storage controller 36 controls the storage memory 22 to store information indicating that the total of the duration time has reached the first set time and data of the duration time (S32). In this case, the total of the duration time may be displayed. Similarly, the second determiner 35 determines whether the total of the duration time in the predetermined time limit has reached the target time (S31). If it is not determined that total of the duration time has reached the target time (NO in S31), the process returns to S22, and it is determined whether the measured heart rate is greater than or equal to the target heart rate. On the other hand, if it is determined that the total of the duration time has reached the target time (YES in S31), controls is performed such that information indicating that the total of the duration time has reached the target time is stored in the storage memory 22 (S27). The subsequent steps S28 and S29 are similar to those described above.

With this configuration, even if the duration time does not reach the target time, as long as the duration time has reached the first set time, the user can easily visually recognize that the duration time has reached the first set time. Thus, the user can facilitate brain function improvement by trying to perform the same constant exercise in a daily life continuously or without interruption to approach the target time. The same holds for the second set time.

In this manner, even if the duration time does not reach the target time, the duration time is added and displayed, for example, so that it is possible to visually appeal that the user has performed an effective exercise leading to brain function improvement. This can lead to facilitating brain function improvement through an exercise within a predetermined time limit.

Such a routine is performed at predetermined intervals, for example, every week. For example, the target time is set at 120 minutes, and the set time is divided into two types such that the first set time is 30 minutes and the second set time is 5 minutes. If a condition is set such that the first set time of 30 minutes needs to be achieved at least twice a week, the display on the display screen facilitates the user to perform an exercise including at least two 30-minute exercises per week, resulting in a total of 120 minutes of exercise. In this case, the total of 120 minutes may also be achieved by performing four 30-minute exercises (30 minutes×4=120 minutes) or by combining two 30-minute exercises (30 minutes×2) with twelve 5-minute exercises (5 minutes×12) each of which is the second set time, adding up to 120 minutes of exercise. In the case of performing a single 90-minute exercise exceeding the first set time, this exercise may be combined with one 30-minute exercise, that is, one instance of the first set time, within the same week to achieve a total of 120 minutes for the week.

### Second Embodiment

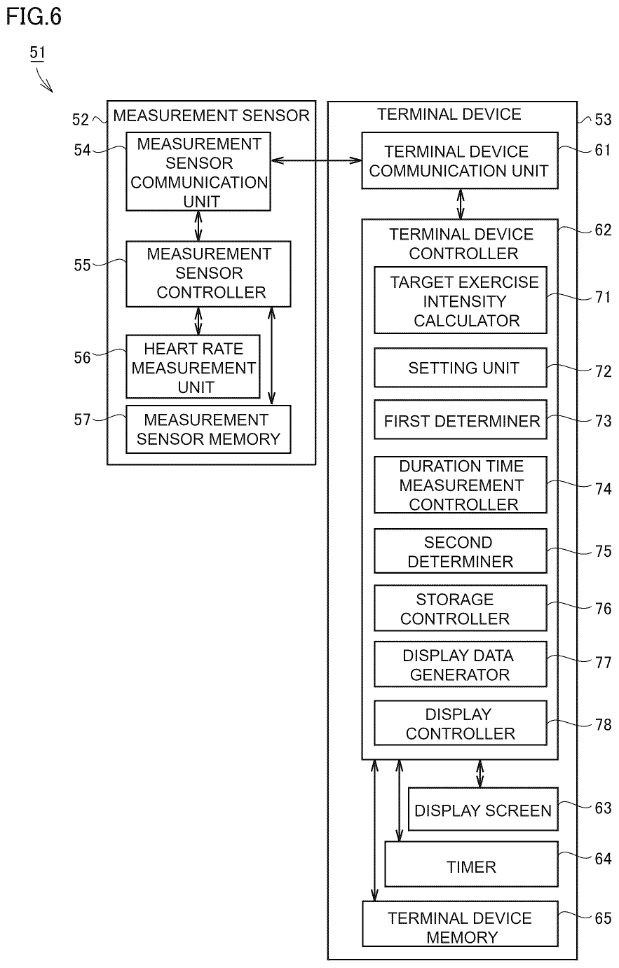

A second embodiment as another embodiment will now be described. FIG. 6 is a block diagram illustrating a brain function improvement facilitating system according to the second embodiment. The brain function improvement facilitating system according to the second embodiment is different from the brain function improvement facilitating device 11 according to the first embodiment in including a measurement sensor that measures a heart rate and a terminal device.

Referring to FIG. 6, a brain function improvement facilitating system 51 is a brain function improvement facilitating system that facilitates improvement of brain function of a user. The brain function improvement facilitating system 51 includes a measurement sensor 52 that is attachable to the body of the user and measures a heart rate of the user over time, and a terminal device 53 communicable with the measurement sensor 52. The measurement sensor 52 includes a measurement sensor communication unit 54 that communicates with the outside, a measurement sensor controller 55 that controls the measurement sensor 52 itself, a heart rate measurement unit 56 that measures a heart rate, and a measurement sensor memory 57 as a storage. The terminal device 53 includes a terminal device communication unit 61 that communicates with the outside, a terminal device controller 62 that controls the terminal device 53 itself, a display screen 63, a timer 64 that measures a time, and a terminal device memory 65 as a storage for storing data. The terminal device controller 62 includes: a target exercise intensity calculator 71 that calculates a target heart rate set as a target exercise intensity necessary for facilitating brain function improvement for the user and a target time to be reached within a predetermined time limit; a setting unit 72 that sets the target heart rate and the target time set by the target exercise intensity calculator 71; a first determiner 73 that determines whether the heart rate of the user measured by the heart rate measurement unit 56 is greater than or equal to the target heart rate set by the setting unit 72; a duration time measurement controller 74 that controls the timer 64 to measure a duration time in which the heart rate of the user is greater than or equal to the target heart rate if the first determiner 73 determines that the heart rate of the user is greater than or equal to the target heart rate; a second determiner 75 that determines whether a total of the duration time measured within a predetermined time limit has reached the target time set by the setting unit 72; a storage controller 76 that controls the terminal device memory 65 to store information indicating that the duration time has reached the target time if the second determiner 75 determines that the duration time has reached the target time; a display data generator 77 that generates display data to be displayed on the display screen 63 based on reaching the target time if the second determiner 75 determines that the duration time has reached the target time; and a display controller 78 that controls the display screen to display at least one of the target heart rate calculated by the target exercise intensity calculator 71, actual related data and time, or the display data generated by the display data generator 77.

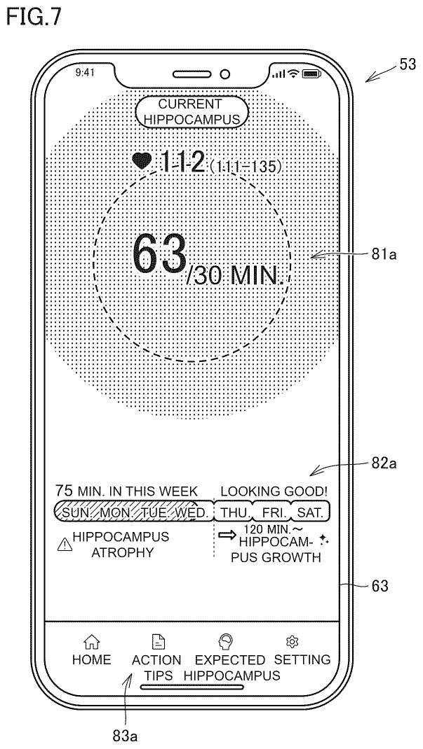

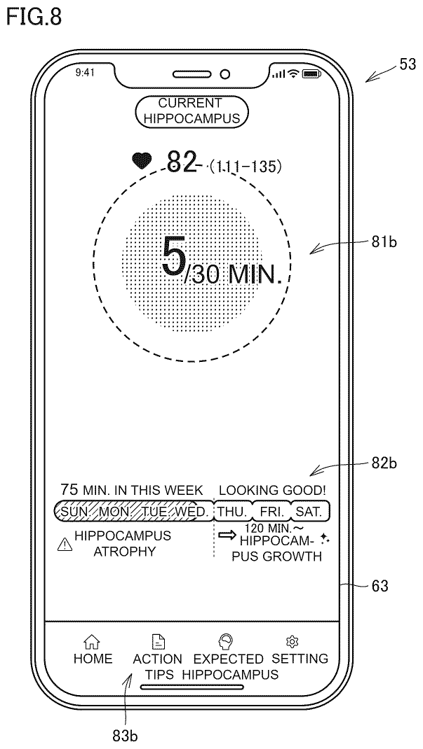

Each of FIGS. 7 and 8 is a schematic view illustrating an example of display data displayed on the display screen 63. First, referring to FIG. 7, the display screen 63 displays, in addition to various data displays, region data 81a visually indicating the size of hippocampus as the size of a circle, graph data 82a indicating, for example, an achieved exercise intensity corresponding to the day of this week, icon data 83a for operation, and other data. The user visually recognizes especially the size of the circle of the region data 81a shown in FIG. 7 and is motivated to exercise. Referring to FIG. 8, the display screen 63 displays, in addition to various data displays, region data 81b visually indicating the size of hippocampus as the size of a circle, graph data 82b indicating, for example, an achieved exercise intensity corresponding to the day of this week, icon data 83b for operation, and other data. The user visually recognizes especially the smallness of the circle of the region data 81b shown in FIG. 8 and is motivated to exercise.

The brain function improvement facilitating system 51 described above can easily facilitate improvement of brain function appropriate for the user.

### Other Embodiments

In the embodiments described above, if the first determiner does not determine that the heart rate of the user measured by the heart rate measurement unit is greater than or equal to the target heart rate within a certain period of the time measured by the timer from a predetermined timing, the display data generator may generate display data indicating that the first determiner does not determine that the heart rate of the user measured by the heart rate measurement unit is greater than or equal to the target heart rate. This configuration makes it possible to visually appeal to the user that the user does not perform an exercise in which the heart rate is greater than or equal to the target heart rate for a certain period of time. Accordingly, it is possible to facilitate the user to perform an aerobic exercise with a heart rate greater than or equal to the target heart rate in a daily life.

In the embodiments described above, determination by the first determiner may be refreshed at each predetermined timing. This configuration makes it possible to effectively facilitate repetitive certain aerobic exercises in a short period such as one week or a long period such as several months.

In the embodiments described above, the brain function improvement may include at least one of suppression of progress of dementia, suppression of atrophy of brain morphology, or improvement of a brain health score. The certain aerobic exercise described above is significantly effective for suppressing the progress of dementia, suppressing atrophy of brain morphology, and improving the brain health score.

A brain function improvement facilitating method according to the present disclosure is a brain function improvement facilitating method for facilitating improvement of brain function of a user, and the method includes: measuring a heart rate of the user over time; calculating a target heart rate and a target time, the target heart rate being set as a target exercise intensity necessary for facilitating brain function improvement for the user, the target time being to be reached within a predetermined time limit; setting the calculated target heart rate and target time; determining whether the measured heart rate of the user is greater than or equal to the target heart rate; performing control to measure a duration time in which the heart rate of the user is greater than or equal to the target heart rate if it is determined that the heart rate of the user is greater than or equal to the target heart rate; determining whether a total of the duration time measured within the predetermined time limit has reached the target time; performing control to store information indicating that the duration time has reached the target time if it is determined that the duration time has reached the target time; generating display data to be displayed based on reaching the target time if it is determined that the total of the duration time has reached the target time; and performing control to display at least one of the calculated target heart rate, actual related data and time, or the generated display data.

This brain function improvement facilitating method can easily facilitate improvement of brain function appropriate for the user.

A brain function improvement facilitating program according to the present disclosure is a program for a brain function improvement facilitating system for use in a brain function improvement facilitating system, the system includes a measurement sensor attachable to a body of a user and including a heart rate measurement unit that measures a heart rate of the user over time, and a terminal device including a storage that stores data, a display screen that displays data, and a timer that measures a time, the terminal device is communicable with the measurement sensor, and the program causes the terminal device to function as: a target exercise intensity calculator that calculates a target heart rate and a target time, the target heart rate being set as a target exercise intensity necessary for facilitating brain function improvement for the user, the target time being to be reached within a predetermined time limit; a setting unit that sets the target heart rate and the target time calculated by the target exercise intensity calculator; a first determiner that determines whether the heart rate of the user measured by the heart rate measurement unit is greater than or equal to the target heart rate set by the setting unit; a duration time measurement controller that controls the timer to measure a duration time in which the heart rate of the user is greater than or equal to the target heart rate if the first determiner determines that the heart rate of the user is greater than or equal to the target heart rate; a second determiner that determines whether a total of the duration time measured within the predetermined time limit has reached the target time set by the setting unit; a storage controller that controls the storage to store information indicating that the duration time has reached the target time if the second determiner determines that the duration time has reached the target time; a display data generator that generates display data to be displayed on the display screen based on reaching the target time if the second determiner determines that the total of the duration time has reached the target time; and a display controller that controls the display screen to display at least one of the target heart rate calculated by the target exercise intensity calculator, actual related data and time, or the display data generated by the display data generator.

This brain function improvement facilitating program can easily facilitate improvement of brain function appropriate for the user.

A storage medium according to the present disclosure is a computer-readable recording medium for use in a brain function improvement facilitating system that facilitates improvement of brain function of a user and includes a measurement sensor attachable to a body of a user and including a heart rate measurement unit that measures a heart rate of the user over time, and a terminal device including a storage that stores data, a display screen that displays data, and a time that measures a time, the terminal device is communicable with the measurement sensor, and the recording medium causes the terminal device to function as: a target exercise intensity calculator that calculates a target heart rate and a target time, the target heart rate being set as a target exercise intensity necessary for facilitating brain function improvement for the user, the target time being to be reached within a predetermined time limit; a setting unit that sets the target heart rate and the target time calculated by the target exercise intensity calculator; a first determiner that determines whether the heart rate of the user measured by the heart rate measurement unit is greater than or equal to the target heart rate set by the setting unit; a duration time measurement controller that controls the timer to measure a duration time in which the heart rate of the user is greater than or equal to the target heart rate if the first determiner determines that the heart rate of the user is greater than or equal to the target heart rate; a second determiner that determines whether a total of the duration time measured within the predetermined time limit has reached the target time set by the setting unit; a storage controller that controls the storage to store information indicating that the duration time has reached the target time if the second determiner determines that the duration time has reached the target time; a display data generator that generates display data to be displayed on the display screen based on reaching the target time if the second determiner determines that the total of the duration time has reached the target time; and a display controller that controls the display screen to display at least one of the target heart rate calculated by the target exercise intensity calculator, actual related data and time, or the display data generated by the display data generator.

This storage medium can easily facilitate improvement of brain function appropriate for the user.

It should be understood that the embodiments disclosed here are illustrative and non-restrictive in every respect. The scope of the present invention is defined by the claims rather than the description above, and is intended to include any modifications within the scope and meaning equivalent to the claims.

### DESCRIPTION OF REFERENCE CHARACTERS

11 brain function improvement facilitating device, 12, 63 display screen, 13 band, 14 button, 21 controller, 22 storage memory, 22, 56 heart rate measurement unit, 24 communication unit, 25, 64 timer, 31, 71 target exercise intensity calculator, 32, 72 setting unit, 33, 73 first determiner, 34, 74 duration time measurement controller, 35, 75 second determiner, 36, 76 storage controller, 37, 77 display data generator, 38, 78 display controller, 41, 42, 43 display data, 51 brain function improvement facilitating system, 52 measurement sensor, 53 terminal device, 54 measurement sensor communication unit, 55 measurement sensor controller, 57 measurement sensor memory, 61 terminal device communication unit, 62 terminal device controller, 65 terminal device memory, 81a,81b region data, 82a, 82b graph data, 83a, 83b icon data.

1. A brain function improvement facilitating device attachable to a body of a user and configured to facilitate brain function improvement of the user, the device comprising:
a storage that stores data;
a display screen that displays data;
a timer that measures a time;
a heart rate measurement unit that measures a heart rate of the user over time;
a target exercise intensity calculator that calculates a target heart rate and a target time, the target heart rate being set as a target exercise intensity necessary for facilitating brain function improvement for the user, the target time being to be reached within a predetermined time limit;
a setting unit that sets the target heart rate and the target time calculated by the target exercise intensity calculator;
a first determiner that determines whether the heart rate of the user measured by the heart rate measurement unit is greater than or equal to the target heart rate set by the setting unit;
a duration time measurement controller that controls the timer to measure a duration time in which the heart rate of the user is greater than or equal to the target heart rate if the first determiner determines that the heart rate of the user is greater than or equal to the target heart rate;
a second determiner that determines whether a total of the duration time measured within the predetermined time limit has reached the target time set by the setting unit;
a storage controller that controls the storage to store information indicating that the total of the duration time has reached the target time if the second determiner determines that the total of the duration time has reached the target time;
a display data generator that generates display data to be displayed on the display screen based on reaching the target time if the second determiner determines that the total of the duration time has reached the target time; and
a display controller that controls the display screen to display at least one of the target heart rate calculated by the target exercise intensity calculator, actual related data and time, or the display data generated by the display data generator.

2. The brain function improvement facilitating device according to claim 1, wherein
if the second determiner does not determine that the duration time has reached the target time, the second determiner determines whether the duration time has reached a first set time shorter than the target time,
if the second determiner determines that the duration time has reached the first set time, the storage controller controls the storage to store information indicating that the duration time has reached the first set time, and
if the second determiner determines that the duration time has reached the first set time, the display data generator generates display data to be displayed on the display screen based on reaching the first set time.

3. The brain function improvement facilitating device according to claim 2, wherein if the second determiner determines that the duration time has reached the first set time, the storage controller controls the storage to store data of the duration time.

4. The brain function improvement facilitating device according to claim 1, wherein the brain function improvement includes at least one of suppression of progress of dementia, suppression of atrophy of brain morphology including a negative change in brain morphology, or improvement of a brain health score.

5. The brain function improvement facilitating device according to claim 1, wherein if the first determiner does not determine that the heart rate of the user measured by the heart rate measurement unit is greater than or equal to the target heart rate within a certain period of the time measured by the timer from a predetermined timing, the display data generator generates display data indicating that the first determiner does not determine that the heart rate of the user measured by the heart rate measurement unit is greater than or equal to the target heart rate.

6. The brain function improvement facilitating device according to claim 1, wherein determination by the first determiner is refreshed at each predetermined timing.

7. The brain function improvement facilitating device according to claim 1, wherein the target exercise intensity calculator calculates the target heart rate using at least one of a rule-based model, a machine learning model, or a deep learning model.

8. The brain function improvement facilitating device according to claim 1, wherein the target exercise intensity calculator calculates, as the target heart rate, an optimized target heart rate that is a heart rate as high as possible while satisfying a condition of performing an exercise continuously or without interruption.

9. A brain function improvement facilitating system that facilitates brain function improvement, the system comprising:
a measurement sensor attachable to a body of the user and including a heart rate measurement unit that measures a heart rate of the user over time; and
a terminal device communicable with the measurement sensor, wherein
the terminal device includes
a storage that stores data,
a display screen that displays data,
a timer that measures a time,
a target exercise intensity calculator that calculates a target heart rate and a target time, the target heart rate being set as a target exercise intensity necessary for facilitating brain function improvement for the user, the target time being to be reached within a predetermined time limit,
a setting unit that sets the target heart rate and the target time calculated by the target exercise intensity calculator,
a first determiner that determines whether the heart rate of the user measured by the heart rate measurement unit is greater than or equal to the target heart rate set by the setting unit,
a duration time measurement controller that controls the timer to measure a duration time in which the heart rate of the user is greater than or equal to the target heart rate if the first determiner determines that the heart rate of the user is greater than or equal to the target heart rate,
a second determiner that determines whether a total of the duration time measured within the predetermined time limit has reached the target time set by the setting unit,
a storage controller that controls the storage to store information indicating that the duration time has reached the target time if the second determiner determines that the duration time has reached the target time,
a display data generator that generates display data to be displayed on the display screen based on reaching the target time if the second determiner determines that the total of the duration time has reached the target time, and
a display controller that controls the display screen to display at least one of the target heart rate calculated by the target exercise intensity calculator, actual related data and time, or the display data generated by the display data generator.

10. A brain function improvement facilitating method for facilitating improvement of brain function of a user, the method comprising:
measuring a heart rate of the user over time;
calculating a target heart rate and a target time, the target heart rate being set as a target exercise intensity necessary for facilitating brain function improvement for the user, the target time being to be reached within a predetermined time limit;
setting the calculated target heart rate and target time;
determining whether the measured heart rate of the user is greater than or equal to the target heart rate;
performing control to measure a duration time in which the heart rate of the user is greater than or equal to the target heart rate if it is determined that the heart rate of the user is greater than or equal to the target heart rate;
determining whether a total of the duration time measured within the predetermined time limit has reached the target time;
performing control to store information indicating that the duration time has reached the target time if it is determined that the duration time has reached the target time;
generating display data to be displayed based on reaching the target time if it is determined that the total of the duration time has reached the target time; and
performing control to display at least one of the calculated target heart rate, actual related data and time, or the generated display data.

11. (canceled)

12. (canceled)

13. The brain function improvement facilitating device according to claim 2, wherein the brain function improvement includes at least one of suppression of progress of dementia, suppression of atrophy of brain morphology including a negative change in brain morphology, or improvement of a brain health score.

14. The brain function improvement facilitating device according to claim 2, wherein if the first determiner does not determine that the heart rate of the user measured by the heart rate measurement unit is greater than or equal to the target heart rate within a certain period of the time measured by the timer from a predetermined timing, the display data generator generates display data indicating that the first determiner does not determine that the heart rate of the user measured by the heart rate measurement unit is greater than or equal to the target heart rate.

15. The brain function improvement facilitating device according to claim 2, wherein determination by the first determiner is refreshed at each predetermined timing.

16. The brain function improvement facilitating device according to claim 2, wherein the target exercise intensity calculator calculates the target heart rate using at least one of a rule-based model, a machine learning model, or a deep learning model.

17. The brain function improvement facilitating device according to claim 2, wherein the target exercise intensity calculator calculates, as the target heart rate, an optimized target heart rate that is a heart rate as high as possible while satisfying a condition of performing an exercise continuously or without interruption.
